(19) United States
(12) Patent Application Publication
(10) Pub. No.: US 2004/0031069 A1
(43) Pub. Date: Feb. 12, 2004

(54) Cloning endangered and extinct species

(76) Inventors: Philip Damiani, Spencer, MA (US); Robert Lanza, Clinton, MA (US); Jose Cibelli, Holden, MA (US); Michael D. West, Boston, MA (US)

Correspondence Address: CROWELL & MORING LLP, INTELLECTUAL PROPERTY GROUP, P.O. BOX 14300, WASHINGTON, DC, 20044-4300, US

(21) Appl. No.: 10/398,608

(22) PCT Filed: Oct. 5, 2001
(86) PCT No.: PCT/US01/31218

(51) Int. Cl. A01K 67/027

(52) U.S. Cl. 800/14; 800/17; 800/21

(57) ABSTRACT

Approximately 100 species become extinct a day. Despite increasing interest in using cloning to rescue endangered species, successful interspecies nuclear transfer has not been previously described, and only a few reports of in vitro embryo formation exist. Here it is shown that interspecies nuclear transfer can be used to clone an endangered species with normal karyo- and phenotypic development through implantation and the late stages of fetal growth, resulting in a newborn animal.

### CROSS REFERENCE TO RELATED APPLICATION

This application claims priority from U.S. Provisional Application Serial No. 60/238,015, filed Oct. 6, 2000, which is incorporated herein in its entirety.

### FIELD OF INVENTION

The present invention concerns the use of interspecies nuclear transfer in order to clone endangered species, and to re-create members of an extinct species. The invention also concerns methods for making a sexual mate for an animal of an endangered or extinct species using chromosome shuffling techniques, as well as methods for correcting chromosomal abnormalities in donor cells prior to nuclear transfer.

### TECHNICAL BACKGROUND

Approximately 100 species become extinct per day. Extinction threatens 11 percent of birds, 25 percent of mammals, and 34 percent of fish species (Porter, 2000). Given current trends, many rare or endangered vertebrate species will soon be lost despite efforts to maintain biodiversity via habitat and wildlife conversation. Even when a species is not endangered or threatened, the loss of biological diversity may lead to extinction of subspecies and other valuable genetic populations (Corley-Smith and Brandhorst, 1999).

The current method of preserving genetic diversity of endangered species in captivity is through a series of captive propagation programs. However, these programs are not without limitations, which include limited physical space for animals, problems with animal husbandry and general reproductive failure of the animal (Lasley et al., 1994). Recent advances in assisted reproductive techniques such as cryogenics of gametes/embryos, artificial insemination and embryo transfer have allowed for new methods for the further propagation of endangered species.

Most recently, there is growing scientific and public interest in using nuclear-transfer techniques to facilitate the rescue of endangered species, or even to restore them after the extinction of intact organisms. However, unlike the cloning of rodents and domestic animals where there is a ready supply of oocytes and surrogate animals, the cloning of highly endangered or extinct species will require the use of an alternative method of cloning known as interspecies nuclear transfer.

Recent in vitro studies have confirmed the ability of bovine oocyte cytoplasm to support mitotic cell cycles under the direction of differentiated somatic cell nuclei of several mammalian species (Dominko et al, 1999; Lanza et al, 1999 a,b). Nuclear transfer units between sheep, pigs, monkeys and rats and enucleated bovine oocytes, all underwent transition to interphase accompanied by nuclear swelling and further progression through the cell cycle as evidenced by successive cell division and formation of a blastocoele cavity at the time appropriate for the species of the donor nuclei. As in other studies, in which nuclei from human somatic cells were transferred into enucleated bovine oocytes, some of the interspecific embryos progressed further and also developed to advanced embryonic stages (Lama et al, 1999 a,b). Furthermore, an attempt at interspecies nuclear transfer with ovine oocytes (Ovis aries) and somatic cells from the argali wild sheep (Ovis ammon) resulted in the production of a few blastocysts. Following embryo transfer fluid accumulation was observed in one recipient via ultrasonography although no fetus or heartbeat was detected (White et. al., 1999).

To date, there has been no evidence that fetal development will result after the fusion of mammalian somatic cells with enucleated xenogenic oocytes. While nuclear transfer between donor and recipient cells of different breeds has been shown to be successful (Well et al., 1998), it is unclear whether recipient cells of completely different species can support the growth and differentiation of a donor nucleus. It is particularly unclear whether the cloned progeny would harbor the mitochondrial DNA (mtDNA) genotype of the recipient cytoplast, as with Dolly the sheep (Evans et al, 1999), and whether mitochondria from a surrogate species are capable of supporting normal embryonic and fetal development.

Here we show that interspecies nuclear transfer can be used to clone an endangered species with normal karyo- and phenotypic development through implantation and fetal development, to generate a newborn animal. Somatic cells from a gaur bull (Bos gaurus), a large wild ox on the verge of extinction, (Species Survival Plan<100 animals) were electrofused with enucleated oocytes from domestic cows to generate the first cloned cross-species animals. Microsatellite marker and cytogenetic analyses confirmed that the nuclear genome of the cloned animals was gaurus in origin. The gaur nuclei were shown to direct normal fetal development, with differentiation into complex tissue and organs and generation of a newborn animal, even though the mtDNA within all the tissue types evaluated was derived exclusively from the recipient bovine oocytes. These results suggest that somatic cell cloning methods could be used to restore endangered, or even extinct, species and populations.

### SUMMARY OF INVENTION

The present invention encompasses methods of cloning endangered or extinct animals, comprising, for instance, the steps:

(1) isolating a somatic cell from an endangered or extinct animal;

(2) transferring the nucleus from said somatic cell into an enucleated suitable recipient cell;

(3) activating said nuclear transfer unit;

(4) implanting said nuclear transfer unit into a suitable surrogate female; and

(5) allowing said nuclear transfer unit to develop to at least the fetal stage, thereby generating a clone of said endangered or extinct animal.

Suitable recipient cells include any cell from a non-endangered animal that supports reprogramming of a somatic cell nucleus back to the one-cell embryo stage, wherein such cells are of a different species than the donor cell from the endangered or extinct animal. Thus, the resulting cloned animals are the result of interspecies or cross-species nuclear transfer.

In methods of cloning extinct species, the resulting cloned animals may then be used to re-create other members of an extinct species, whereby male clones are bred with female clones to re-create members of the species. In cases where cells are not available for cloning from both sexes of the extinct species, the present invention includes methods to generate such cells in order to produce a sexual mate of an extinct animal, for instance by:

(1) isolating a somatic cell from said extinct animal;

(2) removing the sex chromosome from said somatic cell;

(3) inserting the alternative sex chromosome from a non-isogenic animal; and

(4) using nuclear transfer to create an autosomally isogenic, sexually non-isogenic sexual mate of said extinct animal.

Alternative sex chromosomes for methods of producing sexual mates may be isolated from an allogeneic somatic cell of the same extinct species that otherwise could not be used as a nuclear transfer donor, i.e., due to damage of other chromosomes. Alternatively, it may be possible to use the sex chromosome from a xenogeneic cell, particularly if the xenogeneic cell is from a species that is closely related to the extinct animal. Such alternative sex chromosomes may be introduced via microsome mediated chromosome transfer. Microsome mediated transfer may also be used to introduce other chromosomes, i.e., following repair or transfection.

The present invention also encompasses an improved method for preserving and propagating an endangered species that reproduces poorly in zoos until restoration of its habitat is complete, comprising:

(1) isolating a somatic cell from an animal of said endangered species;

(2) transferring the nucleus from said somatic cell into an enucleated suitable recipient cell;

(3) activating said nuclear transfer unit;

(4) implanting said nuclear transfer unit into a suitable surrogate female; and

(5) allowing said nuclear transfer unit to develop into a clone of said endangered animal.

Such a method is performed with the goal of possibly introducing the cloned animal into the restored habitat. The materials used in this methodology can be frozen or preserved at any stage prior to implantation, i.e., to provide a cryobank of somatic cells or embryonic cells to be used to regenerate the species.

### BRIEF DESCRIPTION OF DRAWINGS

FIG. 1. In protocol for Percoll separation of somatic cells from semen, diagram depicting Percoll layers prior to (A) and following (B) centrifugation.

FIG. 2. Cytogenetic and microsatellite analysis of cloned fetuses. a, Standard Geimsa-banded karyotype of a male bovine (Bos taurus) displaying 60 chromosome homologues aligned largest to smallest. b, Standard Geimsa-banded karyotype of a male gaur (Bos gaurus) displaying 58 chromosome homologues aligned largest to smallest. c-e, Geimsa-banded karyotypes of the three cloned fetuses displaying the 58 chromosome homologues, further indicating their gaurus nuclear origin.

FIG. 3. Embryos derived from cross-species nuclear transfer. a, Gaur embryos at the blastocyst stage of development following 7 days of in vitro culture, prior to embryo transfer (100×magnification). b, Hatching blastocysts derived from cross-species nuclear transfer (200×magnification).

FIG. 4. Representation of nuclear transfer-derived fetuses. a, Cloned fetus removed at 46 days of gestation. b-c, Cloned fetuses removed at 54 days of gestation. d, Normal growth curve of bovine fetuses, (adapted from Evans and Sachs, 1973). Dashed line represents crown rump length of beef breeds of cattle. Solid line represents crown rump length of dairy breeds of cattle. •, Crown rump lengths of the nuclear transfer-derived fetuses.

FIG. 5. Ultrasound images of gaur fetus at 80 days of gestation. (a) Longitudinal cross section of the cranium, displaying the frontal bones (skull), maxillary (mouth) and the orbits (eye). (b) Longitudinal cross section of the posterior region displaying hindlimb and umbilicus.

FIG. 6. Microsatellite analyses of bovine and gaur fibroblast cell lines assayed with bovine chromosome 21 specific probe. All four cloned fetuses (3 electively removed fetuses, and the fetus recovered following late-term abortion at 202 days) were derived from gaurus nuclear DNA. Bovine fibroblast (B), donor gaur fibroblast (G) and fetal gaur fibroblast (F).

FIG. 7. Ethidium bromide stained agarose gel of restriction digests of bovine and gaur mtDNA. Total DNA was isolated from adult bovine and gaur fibroblast cells and the D-loop region of mtDNA amplified. The D-loop regions of fetal gaur mtDNA were amplified from DNA isolated from twelve tissue types (1-12; brain, eye, tongue, bone, heart, intestine, liver, kidney, gonad, muscle, skin, hoof). The amplified fragments of mtDNA were digested with restriction enzyme BstNI, and electrophoresed through an agarose gel. mtDNA analyses revealed that all tissue types derived from cloned fetuses were Bos taurus in origin and had undetectable levels of gaurus mtDNA. U, undigested PCR fragment; D, digested fragment.

FIG. 8. Interspecific clones do not retain the nuclear cognate mtDNA. Total DNA was extracted from bovine fibroblasts, gaur fibroblasts, and tissues (brain, liver and skeletal muscle) from the three cloned fetuses (Fetus 1-3) and used to amplify a 483 by fragment corresponding to the mtDNA D-loop region. This fragment was labeled with [³²P]a-dCTP in the last cycle of the PCR (Cibelli et al, 1998), digested with ScrFI (a) or SphI (b) or and electrophoresed through a 10% polyacrylamide gel. Phosphorimage analyses showed that the three different tissues from the three fetuses have undetectable levels of gaur mtDNA. U, undigested PCR fragment; D, digested with respective restriction endonucleases. Molecular weights are shown on the left of each panel.

FIGS. 9A-D. Photographs of Noah, a newborn gaur produced by interspecies nuclear transfer, after birth.

### DETAILED DESCRIPTION OF THE INVENTION

The present invention includes a method of cloning an endangered or extinct animal, comprising:

(1) isolating a somatic cell from an endangered or extinct animal or the nucleus from such a cell;

(2) transferring the somatic cell or nucleus from said somatic cell into an enucleated suitable recipient cell;

(3) activating said nuclear transfer unit;

(4) implanting said nuclear transfer unit into a suitable surrogate female; and

(5) allowing said nuclear transfer unit to develop to at least the fetal stage, thereby generating a clone of said endangered or extinct animal.

Suitable recipient cells include any cell from a non-endangered animal that supports reprogramming of a somatic cell nucleus back to the one-cell embryo stage, i.e., enucleated oocytes, wherein such cells are of a different species than the donor cell from the endangered or extinct animal. Thus, the resulting cloned animals are the result of interspecies or cross-species nuclear transfer.

Preferred endangered animals to benefit from the present invention include the gaur, African bongo antelope, Sumatran tiger, giant panda, Indian desert cat, mouflon sheep and rare red deer. Suitable recipient cells for reprogramming would be chosen from a closely related animal that has a similar gestation period and species size. For instance, where the endangered animal to be cloned is gaur, a suitable recipient cell would be an enucleated bovine oocyte. Where the endangered animal to be cloned is an African bongo antelope, a suitable surrogate female is an eland. Where the endangered animal to be cloned is an Indian desert cat, a Sumatran tiger, or a cheetah, a suitable surrogate female is a domestic cat. Where the endangered animal to be cloned is a Giant panda, a suitable surrogate female is an American black bear, and a suitable recipient cell is an enucleated American black bear oocyte. Where the endangered animal to be cloned is a mouflon sheep, a suitable surrogate female is a domestic sheep. Where the endangered animal to be cloned is a rare red deer, a suitable surrogate female is a common white tailed deer.

The invention includes cloned animals made by the methods described herein, wherein such animals include embryos, blastocysts, fetuses and animals that develop to at least the neonatal stage, as well as adult cloned animals. The invention also includes cells and tissues formed according to the claimed methodology, for use in transplantation therapy of endangered animals.

Any somatic cell from an endangered or extinct animal may be used as a donor cell of the present invention. Such cells may be frozen prior to use as nuclear donors, or preserved by any other means, i.e., in alcohol. Cell nuclei may also be preserved rather than whole cells. In one embodiment of the present invention, the frozen cells are isolated from semen, which the present inventors have surprisingly found to be a source of somatic cells suitable for use as donors for nuclear transfer.

Extinct animals may also be cloned using the methods herein. A preferred extinct animal to be cloned is a bucardo mountain goat of Spain, wherein a suitable recipient cell is an enucleated oocyte from a domestic goat, and a suitable surrogate female is a domestic goat. Where both male and female sources of cells are available to be used as donors for nuclear transfer, both male and female animals may be cloned and bred to re-generate more members of an extinct species. Such a method comprises, for instance:

(1) using nuclear transfer from frozen somatic cells to clone a male animal of an extinct species;

(2) using nuclear transfer from frozen somatic cells to clone a female animal of an extinct species; and

(3) breeding said male clone with said female clone to re-create members of said extinct species.

Where sources for both male and female counterparts of an extinct species do not exist, the present invention also includes methods for producing a sexual mate for a single clone of an extinct animal, comprising:

(1) isolating a somatic cell from said extinct animal;

(2) removing the sex chromosome from said somatic cell;

(3) inserting the alternative sex chromosome from a non-isogenic animal; and

(4) using nuclear transfer to create an autosomally isogenic, sexually non-isogenic sexual mate of said extinct animal.

This method may be used to create an autosomally isogenic, sexually non-isogenic animal mate for an extinct or endangered animal. In this embodiment, particularly for extinct animals, the somatic cell may need to be isolated from a sample of frozen cells. In cases where an animal is endangered or nearing endangered levels, somatic cells, preferably semen cells, may be frozen in preparation for the methodology of the invention. Where the animal is extinct and frozen cells for replacement chromosomes do not exist, the alternative chromosome may be taken from a xenogeneic animal, preferably one that is closely related to the extinct animal. In this regard, copending application Ser. No. ______ pertains specifically to the making of cloned breeding pairs and particularly autosomally isogenic, sexually non-isogenic breeding pairs using chromosome shuffling techniques, and is herein incorporated by reference in its entirety.

Also encompassed are methods of eliminating chromosomal abnormalities from the clone of an animal wherein a damaged chromosome from a somatic cell is removed or programmed for removal, and a non-damaged chromosome from a non-isogenic animal is inserted. Nuclear transfer is then used to create an animal, embryo, blastocyst, fetus or cell from said chromosomally corrected somatic cell.

In order to replace a sex chromosome or autosome, the original chromosome must be removed. When a sex chromosome is removed according to the present invention, it may be either an X or a Y chromosome, and it may be replaced by the alternative sex chromosome from a non-isogenic allogeneic animal, or an a non-isogenic, xenogeneic animal. In the case where the somatic cell of interest is from a male animal, the Y chromosome may be replaced by the X chromosome from another copy of the somatic cell to yield a cell with two X chromosomes. The chromosome to be replaced may be removed by any feasible technique. For instance, the unwanted chromosome may be removed by targeting by homologous recombination a gene or DNA sequence that results in loss of the chromosome upon mitosis or meiosis. As discussed in U.S. Pat. Nos. 5,270,201 and 6,077,697, chromosomal instability results when sequences are introduced which function as a centromere. Such sequences cause a dicentric chromosome to be created, which undergoes breakage potentially leading to loss of the chromosome during cell division. Loss of chromosomes that have been genetically modified with additional centromeric sequences can be detected by karyotype analysis. Cells which lose the targeted chromosome may be also be selected by including a negative selectable marker such as thymidine kinase whereby cells retaining the chromosome or pieces of the chromosome will not survive under selective conditions (i.e., gancyclovir in the case of thymidine kinase).

An advantage of using somatic cells as nuclear donors is that they may be expanded readily in culture prior to chromosome shuffling techniques. However, embryonic cells may also be used, as may the nuclei of somatic cells, which are advantageous in that they may be maintained in a preservative (such as alcohol) prior to nuclear transfer, i.e., stored for future use. Preferred somatic cells will be proliferating, i.e., in a proliferative state, but need not necessarily be expanded in culture. The somatic cells may be genetically altered in other ways prior to or subsequent to chromosome exchange. For instance, said cells may be modified with a chromosomal insertion or deletion, where a transgenic animal is desired that produces specific proteins in its bodily fluids or mammary glands, or where it is desirable to remove or mutate genes involved in xenotransplantation rejection. The alternative sex chromosome to be introduced may also be genetically altered from its native state.

The chromosomes to be inserted according to the claimed methods may be inserted via microcell-mediated chromsome transfer, or any other suitable technique known in the art, e.g., via injection. Methods for the preparation and fusion of microcells containing single chromosomes are well known. See, e.g., U.S. Pat. Nos. 5,240,840; 4,806,476; 5,298,429 (herein incorporated by reference in their entirety; see also Fournier, 1981, Proc. Natl. Acad. Sci. USA 78: 6349-53; Lambert et al., 1991, Proc. Natl. Acad. Sci. USA 88: 5907-59; Yoshida et al., 1994, J. Surg. Oncol. 55:170-74; Dong et al., 1996, World J. Urol. 14: 182-89. Chromosomes to be introduced into cloned cells or cells to be cloned will preferably include a selectable marker, such as aminoglycoside phosphotransferase, for example, so that cells receiving the chromosome via microcell fusion may be readily selected from those that do not. In this regard, Siden and colleagues describe the construction of a panel of four microcell hybrids containing four separate insertions of the exogenous neomycin resistance gene into mouse chromosome 17. See Siden et al., 1989, Somat. Cell Mol. Genet. 15(3): 245-53.

U.S. Pat. No. 6,133,503 also describes methodology for producing microcells by treating a host donor cell with a mitotic spindle inhibitor such as colchicine, which results in the formation of micronuclei, then with cytochalasin B, which results in the extrusion of microcells which contain one or a few chromosomes. The methods of U.S. Pat. No. 5,635,376 are also helpful in the context of the present invention, in that this patent provides for female muntjac cell lines in which there is, for example, a ten-fold difference in chromosomal size between the diploid muntjac chromosomes and human chromosome 11. Thus, these female muntjac cell lines are useful for the amplification of desired chromosomes prior to use in cells to be cloned because desired chromosomes may be purified to apparent homogeneity from the resulting hybrids using conventional equipment given the large size difference between the chromosome of interest and the muntjac chromosomes. These patents are herein incorporated by reference in their entirety.

The cloned animals, embryos, blastocysts, fetuses and cells produced by the methods described herein are also part of the invention, as are the sexual mates and breeding pairs produced and their offspring. Also included are the individual replacement chromosomes used for the present invention and any DNAs used to make genetic modifications, as well as any intermediary cell lines such as muntjac cell lines used to amplify the desired replacement chromosomes.

Microcell-mediated chromosome transfer may also be used to correct chromosomal abnormalities in the cells of an extinct animal, comprising:

(1) isolating a somatic cell from a frozen sample of cells from an extinct animal;

(2) removing at least one damaged chromosome from said isolated somatic cell;

(3) inserting a functional non-isogenic chromosome into said isolated cell; and

(4) using nuclear transfer to create a partial clone of said extinct animal.

Such a method finds particular utility when there are limited frozen cells from an extinct source, and such cells contain damaged DNA as to render them unsuitable as donors for nuclear transfer. Thus, rather than using the entire source of frozen cells in repeated failed attempts to create nuclear transfer embryos, genetic dissection of a single cell could be performed in order to correct any chromosomal deficiencies. Again, chromosomes to be used for repair of damaged genomes may be taken from a separate sample of frozen cells taken from an allogeneic extinct animal, or from the same sample of cells. Alternatively such cells may be taken from a xenogeneic animal, thereby creating a partial clone that is a hybrid of two species.

Some researchers have argued against the cloning of endangered species, alleging that the practice could overshadow efforts to preserve habitat. However, the ability to accomplish interspecies nuclear transfer offers the possibility of keeping the genetic stock of endangered and extinct species on hand without maintaining a large quantity of species in captivity, which is a costly endeavor particularly with regard to large animals. Maintaining frozen cells as a type of “frozen zoo” offers a type of genetic trust for reconstituting entire populations of a given species when habitat restoration is complete. Thus, the cloning of endangered species can actually be described as an improved method for preserving and propagating an endangered species—particularly those that reproduce poorly in zoos—until habitat restoration is complete, wherein such cells are used in a method comprising:

(1) isolating a somatic cell from an animal of said endangered species;

(2) storing said somatic cell or the nucleus from said cell until habitat restoration is complete;

(3) transferring the nucleus from said somatic cell into an enucleated suitable recipient cell;

(4) activating said nuclear transfer unit;

(5) implanting said nuclear transfer unit into a suitable surrogate female; and

(6) allowing said nuclear transfer unit to develop into a clone of said endangered animal.

Such a method would further include introducing the cloned animal back into the restored habitat once the animal is old enough and the habitat has been prepared or restored.

The following non-limiting examples are representative of what can be accomplished with the methods described herein.

### EXAMPLE 1

### Isolation of Somatic Cells from Semen

The cloning of animals by nuclear transfer has many applications in such diverse fields as agriculture, medicine and the preservation of endangered species. One difficulty commonly faced, however, is an adequate source of somatic cells. In the case of agricultural species such as cattle, highly-valued studs are often lost with no known preservation of the genome for cloning. This invention describes a technique to isolate viable somatic cells from semen, urine, milk and other sources where the isolation of somatic cells is problematic.

While semen is often thought of as being largely a solution of spermatozoa that are haploid, somatic diploid cells may occasionally be shed as well. We centrifuged 0.75 ml of bovine semen at 700×g (45%-90% percoll gradient for 30 minutes), aspirated the supernatant, and resuspended the pellet of 500 ml in DMEM medium with 15 FCS. The resulting cell suspension was then plated in 35 mm² tissue culture plate. The culture dishes were aspirated, washed and refed 24 hours (after and every other day following). After five days of culture, fibroblastic cells were observed attached to the tissue culture dish. These somatic cells can then be propagated, cryopreserved, or used as somatic cell donors for the production of nuclear transfer embryos and calves. An alternative approach would be to use a Fluorescence Cell Sorter machine, which can separate sperm from somatic cells based upon DNA content.

To reduce the chance of spontaneous abortion, fetuses may be extracted at 40 days, and fetal fibroblasts isolated and frozen. From these fetal fibroblasts, the final animals can be cloned. Cells can be isolated in a similar manner from other fluids such as rnilk, blood or urine where such samples have been saved. In addition, such cells can be cultured from frozen tissue such as skin biopsy, skeletal muscle, or whole frozen animals.

The success of this method can be explained perhaps by analyzing the method of semen processing for the purpose of freezing and later use in artificial insemination. During extraction, an artificial vagina is used to collect the ejaculate and perhaps some of the cells that are around the penis along with free somatic cells originating in the accessory glands, ducts and testicle themselves will be mingled with the ejaculate. This technique will allow bulls to be “resurrected” in instances where the bulls are no longer alive but their frozen semen is available. The method is reproduced in detail below:

A. Establishment of Cell Lines from Cryopreserved Semen

NOTE: Please wear gloves for every step of the procedure to prevent cross contamination of samples.

Percoll Separation of Sperm (Performed at Room Temperature)

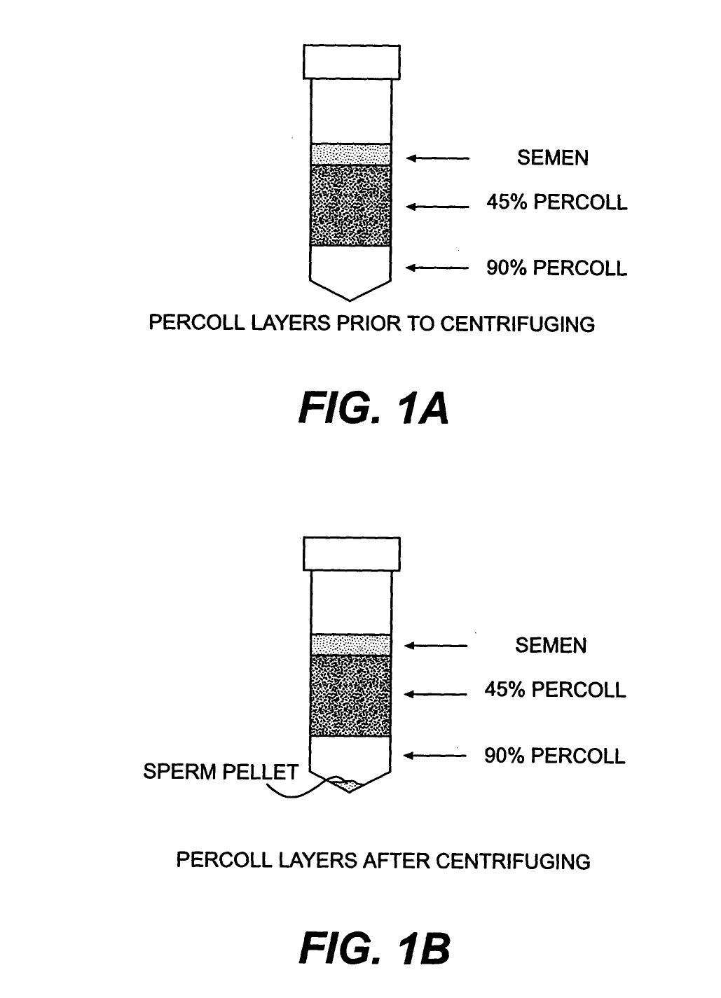

Step 1. In a sterile 15 ml conical centrifuge tube, layer 2 ml 90% Percoll then carefully layer 2 ml of 45% Percoll on top of the 2 ml of 90% Percoll layer as shown in the diagram below. It is best to use either a 1000 ul pipette or a 9 ml pastuer pippete. It is very critical to have a very defined interface between the two layers. This will be observed clearly because the 45% Percoll is pinkish in hue and the 90% Percoll is clear. A very defined interface will be observed if layered correctly (see FIG. 1A).

Step 2: Thaw semen in 35° C. water for 1 min. Record all information from semen straw, including bull name and registration numbers and collection date into your laboratory notebook. Step 3: Thoroughly dry the straw of semen with a KemWipe wet with ethanol and then snip end of semen straw with a clean scissor. Place the open end into a clean 15 ml conical tube. Then carefully snip off the plug end of the straw and deposit all semen into tube.

Step 4: With a 500 ul pipette, carefully layer all of the semen onto the top of the Percoll layers.

Step 5: Centrifuge at 700×g (2000 rpm using a 6.37 inch tip radius) for 30 minutes.

Step 6: After centrifugation, a sperm pellet will be observed at the bottom of the 90% Percoll layer as shown in diagram below (FIG. 1B).

Step 7: Aspirate off the Percoll gradients leaving the sperm pellet in the tip of the tube. This is usually about only 200 ul of pellet (this will vary depending on the number of semen straws thawed).

Step 8: With a clean pipette tip, move the pellet into either a 35 mm tissue culture treated plate or a 4 well Nunc plate with complete DMEM medium.

Step 9: Remove the medium the following day and add fresh medium to the plates.

Step 10: Carefully observe the plates for the presence of cells—this will depend on the semen, usually 7-14 days after the initial plating.

Step 11: Follow standard Cell Culture Techniques once a cell line is observed.

Stock Solutions

45% Percoll Solution

A. Ingredients

1. 1.5 ml 90% Percoll Stock Solution.

2. 1.5 ml Sperm TL with BSA.

B. Procedure

1. Use aseptic techniques.

2. Transfer ingredients to a sterile tube.

3. Invert to mix.

4. Do not attempt to filter.

Sperm TL Without BSA

A. Ingredients

1. 25 ml sperm TL stock.

2. Adjust pH to 7.4 with 1 M HCl.

3. Filter sterilize

4. Prepare daily.

Modified Sperm TL (10×Stock Used to Prepare 90% Percoll)

A. Ingredients

1. 3.09 ml 1M KCl.

2. 2.92 ml 0.1M NaH₂PO₄

3. 4.675 gm NaCl

4. 2.380 gm Hepes

B. Procedure

1. Add prescribed amounts of KCl and NaH₂PO₄ solutions to˜50 ml H₂O in volumetric flask.

2. Add NaCl and Hepes.

3. Adjust water to 100 ml.

4. Adjust pH to 7.3.

5. Filter sterilize and transfer to a glass bottle.

6. Store refrigerated indefinitely.

7. Readjust pH as needed.

1M CaCl₂—Used in Making 90% Percoll

A. Ingredients

1. 735 mg CaCl2*2H₂O.

2. Reagent grade water.

B. Preparation

1. Weigh CaCl₂.

2. Add 5 ml H₂O.

3. Filter sterilize or autoclave.

4. Store in glass bottle indefinitely.

0.1M MgCl₂—Used in Making 90% Percoll

A. Ingredients

1. 20.3 mg MgCl₂*6H₂O.

2. Reagent grade water.

B. Preparation

1. Weigh MgCl₂.

2. Add 10 ml water.

3. Filter sterilize or autoclave.

4. Store in glass bottle indefinitely.

90% Percoll Solution

A. Ingredients

1. 45.0 ml Percoll

2. 5.0 ml Modified Sperm T1 (10×stock)

3. 0.0985 ml 1M CaCl₂

4. 0.197 ml 0.1M MgCl₂

5. 0.184 ml Lactic Acid (60% syrup)

6. 104.5 mg NaHCO₃

B. Procedure

1. Combine ingredients while stirring.

2. Store refrigerated.

3. Do not attempt to filter.

1. SPERM TL STOCK

| | Final | | |
|---|---|---|---|
| Compound | mM | mg/100 ml | mg/500 ml |
| NaCI | 100 | 582 | 2910 |
| KCI | 3.1 | 23 | 115 |
| NaHCO3 | 25 | 209 | 1045 |
| NaH₂PO₄H₂O | 0.29 | 4.1 | 20.5 |
| Hepes | 10 | 238 | 1190 |
| Na Lactate | 21.6 | 368 ul | 1840 ul |
| (60% syrup) | | | |
| Phenol Red | 1 ul/ml | 100 ul | 500 ul |
| CaCl₂2_{H}₂O* | 2.10 | 29 | 145 |
| MgCl₂6H₂O* | 1.5 | 31 | 155 |

*Add last.
Check osmolarity (290-310 mOSM).
Filter into sterile bottle.
Store at 4° C.
Media components derived from: Parrish, J.J., J.L. Susko-Parrish and N.L. First. 1985. Theriogenology 24:537.

Chemical Components

| Sigma | | |
|---|---|---|
| Catalog | | |
| Number | Abbreviation | Name |
| C7902 | CaCl₂*2H₂O | Calcium Chloride-H₂O |
| H3375 | | Hepes |
| M2393 | MgCl₂-6H₂O | Magnesium Chloride-6H₂O |
| P1644 | | Percoll |
| P0290 | | Phenol Red |
| P5405 | KCl | Potassium Chloride |
| S5761 | NaHCO₃ | Sodium Bicarbonate |
| S5886 | NaCl | Sodium Chloride |
| L4263 | | Sodium Lactate (60% syrup) |
| S9638 | Na₂HPO₄*H₂O | Sodium Phosphate |

B. Nuclear Transfer Using Somatic Cells Isolated From Semen

Using the above techniques, we have found that when a single straw of semen is thawed and put in culture under conditions that will favor the growth of epithelial/fibroblast-like cells, colonies can be detected. Using this protocol, we were able to obtain somatic cells from a straw of bull semen, and use those somatic cells to generate embryos by nuclear transfer.

Three replicates of nuclear transfer were performed with three separate Londondale Sperm Cell Lines:

| Cultured | Cleaved | % Cleaved | Blastocysts | % Blastocysts |
|---|---|---|---|---|
| 51 | 26 | 51% | 9 | 18% |
| 191 | 73 | 38% | 37 | 19% |
| 49 | 28 | 57% | 10 | 20% |

6 embryos were transferred into three recipients, but no pregnancy was detected.

One replicate of nuclear transfer was performed with a Whiteleather Mark Sperm Cell Line.

| Cultured | Cleaved | % Cleaved | Blastocysts | % Blastocysts |
|---|---|---|---|---|
| 53 | 18 | 33% | 8 | 15% |

6 Embryos were transferred into 3 recipients—1 pregnancy was detected and is still ongoing (approx 67 days—sexed as male).

C. Characterization of Sperm Cell Lines

Karyotyping

Karyotypes were done on both sperm cell lines; images taken and saved. Results indicate that the cells are of bovine origin and have 60 chromosomes. Samples of NT embryos, cell line, semen and extracted DNA were sent to Celera AgGen for DNA analysis.

Staining of Semen Cell Line

Initial staining of cell lines was performed using alpha tubulin as a general (positive control) marker and Pan Cytokeratin as epithelium marker. Results indicated that there was no staining for the Pan Cytokeratin marker for both concentrations used. Alpha tubulin positive control worked (images not shown). This suggests that the cells are not of epithelial nor endothelial origin, and are probably fibroblasts.

### EXAMPLE 2

### Cloning of Endangered Species

Materials and Methods

Adult Gaur Cell Line Derivation

Dermal fibroblasts were isolated from an adult male gaur (Bos gaurus) at post mortem. A skin biopsy was minced and cultured in DMEM supplemented with 15% fetal calf serum, L-glutamine (2 mM), non-essential amino acids (100 μM), B mercaptoethanol (154 μM) and antibiotics at 38° C. in a humidified atmosphere of 5% CO₂ and 95% air. The tissue explants were maintained in culture and a fibroblast cell monolayer established. The cell strain was maintained in culture, passaged twice and cryopreserved in 10% dimethyl sulfoxide (DMSO) and stored in liquid nitrogen. Donor cells were thawed, cultured, passaged and further propagated prior to nuclear transfer.

Nuclear Transfer and Embryo Culture

Bovine (Bos taurus) oocytes were obtained from abattoir-derived ovaries as previously described (Damiani et al, 1996). Oocytes were mechanically enucleated at 18-22 hours post maturation, and complete enucleation of the metaphase plate confirmed with bisBenzimide (Hoechst 33342) dye under fluorescence microscopy. A suspension of actively dividing gaur cells was prepared immediately prior to nuclear transfer. The cell suspension was centrifuged at 800×g and 5 [0.1 of the resulting cell pellet used for the donor cells. A single cell was selected and transferred into the perivitelline space of the enucleated oocyte. Fusion of the cell-oocyte complexes was accomplished by applying a single pulse of 2.4 kV/cm for 15 μsec. Fused complexes were chemically activated and cultured as previously described (Cibelli et al, 1998). Cleavage rates were recorded and development to the blastocyst stage was assessed on days 7 and 8 of culture. Resulting blastocysts were non-surgically transferred into progestrin-synchronized recipients.

Fetal Gaur Cell Line Derivation

To confirm their genomic origin, three fetuses were sacrificed and collected by cesarean section at 46 (n=1) and 54 (n=2; twins) days of gestation. Individual fetuses were placed in a sterile container with Dulbecco's phosphate buffered saline (PBS) supplemented with antibiotic, packed in wet ice and transported to the laboratory. Upon arrival to the laboratory, crown-rump lengths (CRL) were recorded and the external morphology was evaluated for gross abnormalities. The left forelimb from each gaur fetus was aseptically removed, minced and cultured as described above. After 5-10 days confluent fetal fibroblast cell lines were derived. Cell strains were either subjected to microsatellite marker and cytogenetic analyses, or cryopreserved for long-term storage.

Cytogenetic Analysis

Cells were treated with colcemid (0.04 pg/ml) for 20 minutes at 37° C. in an atmosphere of 5% CO₂ and 95% air. Following colcemid treatment, cells were trypsinized and centrifuged for 5 minutes at 200×g and the supernatant removed. Cells were resuspended in a prewarmed hypotonic solution (0.075M KCl) and incubated at 37° C. for 12 minutes. Cells were then centrifuged and the resulting pellet resuspended in 8 ml of Carnoy's fixative (3:1 methanol: glacial acetic acid) at room temperature for 30 minutes. Fixed cells were centrifuged and washed twice in fresh Carnoy's fixative. After the last centrifugation, the cells were resuspended in 0.5 to 1.0 ml of freshly prepared fixative and single drops were placed on clear slides and air-dried. Slides were stained in a stain solution consisting of Wright's stain and 0.06 M phosphate buffer, pH 6 (1:3 ratio/slide). The entire slide was covered with the stain preparation for 3 minutes, rinsed with distilled water and air-dried. Ten Giemsa-banded cells in metaphase configuration were examined for complete chromosome numbers for each cell line. Five cells in metaphase were photographed and one karyotype constructed and chromosomes arranged in pairs from the largest to smallest.

Mitochondrial DNA and Microsatellite Analysis

Mitochondrial DNA was analyzed using two independent methods. 1) Restriction Fragment Polymorphism. Approximately 0.25 μg of total DNA extracted from different tissues by standard procedures (Moraes, 1992) were used to amplify a 483 bp fragment from the mtDNA D-loop region. Oligonucleotide sequences corresponded to positions 16021-16043 and 165-143 of the Bos taurus mitochondrial genome (GENBANK accession number NC_{—}001567)(Anderson et al, 1982). Although there is variation in the nucleotide sequence between the Bos taurus and Bos gaurus (GENBANK accession number AF083371)(Ward et al, 1999) mtDNA D-loop regions, the oligonucleotide primers have 100% homology with mtDNA from both species. The amplified fragment was labeled with [³²P]-dCTP in the last cycle of the PCR to avoid the detection of heteroduplexes (Moraes et al, 1992). Amplicons were digested with SphI or ScrFI and analyzed by PAGE and Phosphorimaging (Cyclone, Packard Inst.). 2)

Allele Specific PCR

Oligonucleotide primers corresponding to relatively divergent regions of the mtDNA D-loop were used to amplify a 480 by fragment specifically from taurus or gaurus. The gaurus primers were: forward ATAGTACATGAACTCATTAATCG and reverse TTGACTGTAATGCCCATGCC. The taurus primers were: forward CATAATACATATAATTATTGACTG and reverse TTGACTGTAATGTCCATGCT. Amplification were performed with the following cycling program: 94° C. 1′:65° C. 1′:72° C. 1′ for 30 cycles. Microsatellite analysis of the bovine chromosome 21 (D21S18, Research Genetics) was performed by PCR amplification of the marker after end-labeling one of the oligonucleotide primers with [³²P]-ATP. The amplification products were separated in a denaturing polyacrylamide gel electrophoresis, and analyzed by phosphorimaging.

Results

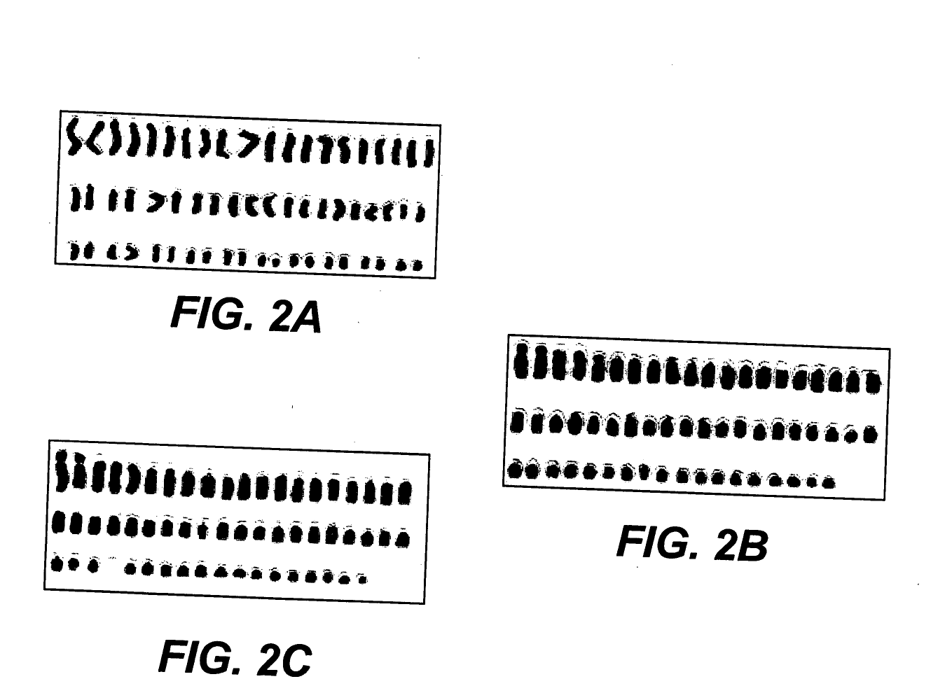

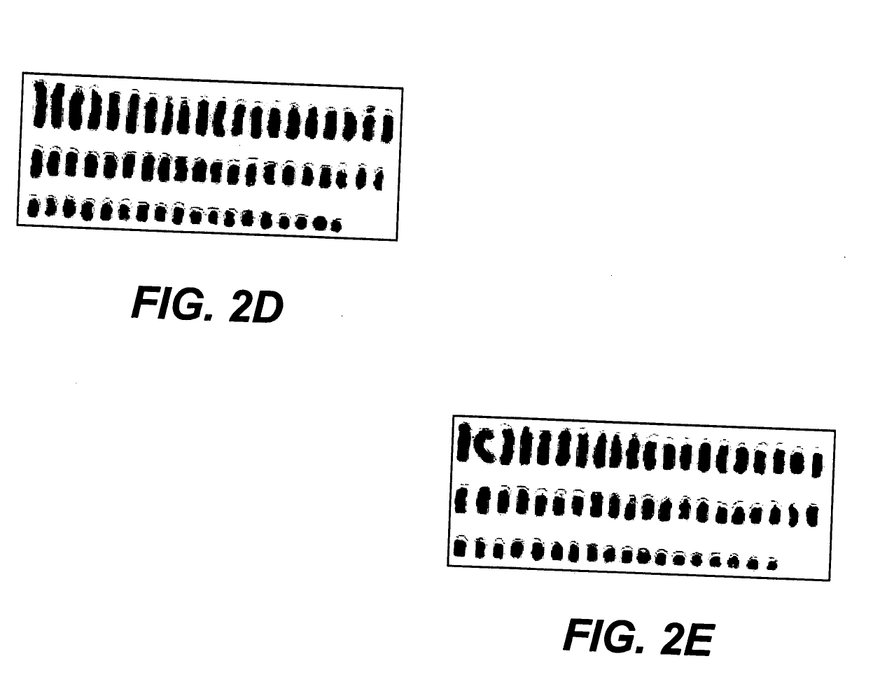

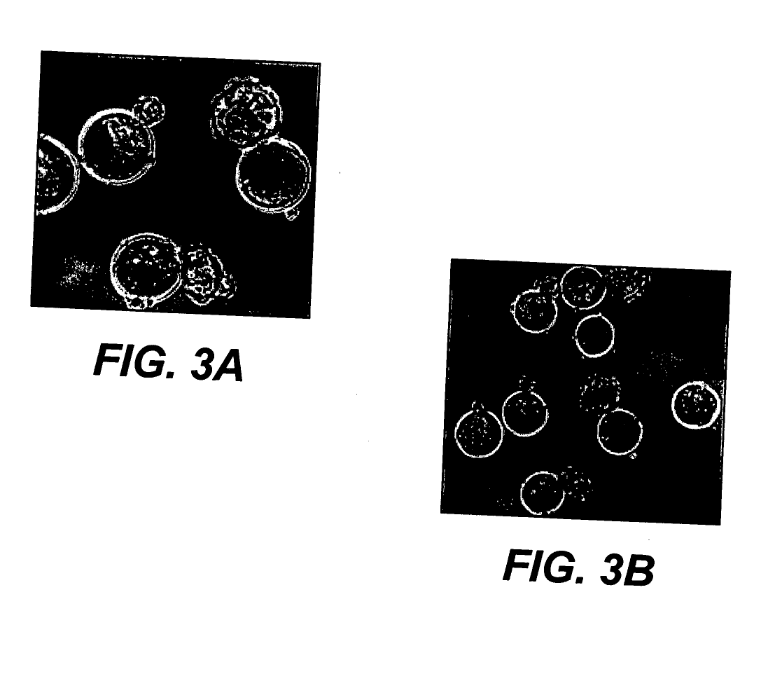

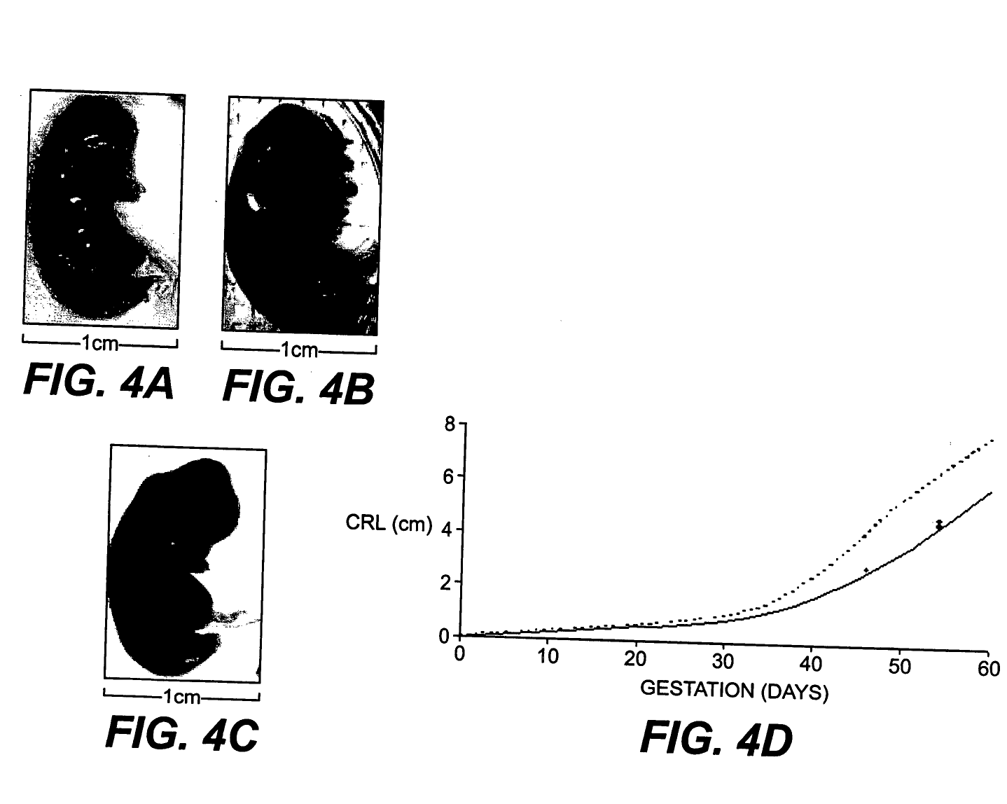

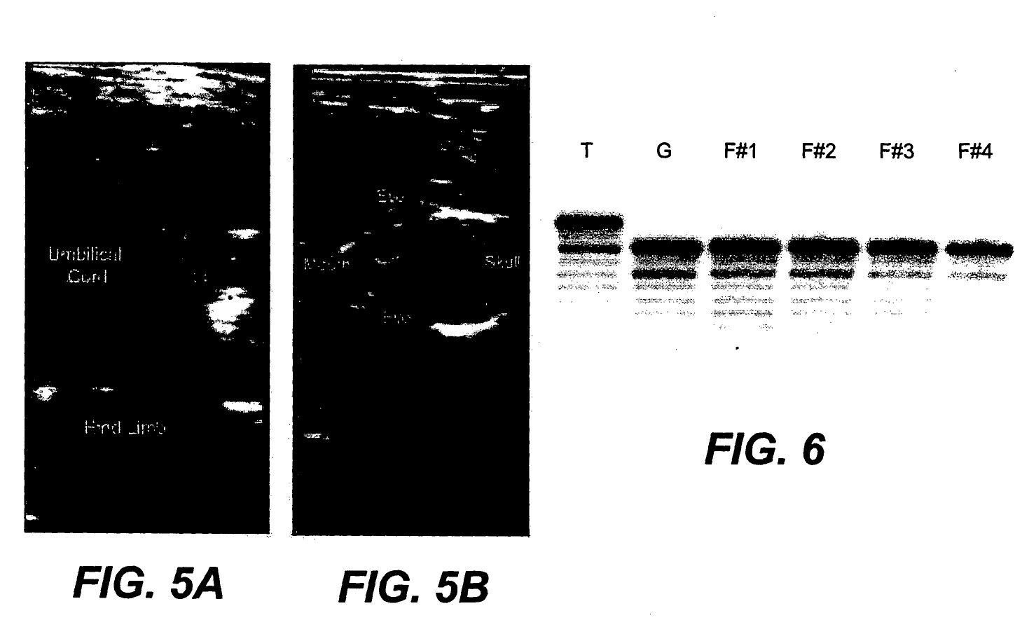

Karyotype analysis of cells derived from the gaur (Bos gaurus) at post mortem revealed a normal diploid chromosome number of 2n=58, made up of a pair of small sex (X and Y) submetacentric chromosomes, two larger submetacentric autosomes (different in size) and 54 acrocentric autosomes, most of which could be arranged in pairs in descending order of size (FIG. 2b). In vitro matured bovine (Bos taurus) MII oocytes were enucleated 18-22 hours after onset of maturation and a total of 692 embryos reconstructed by nuclear transfer using the donor gaur cells as previously described (Cibelli et al, 1998). Eighty-one blastocysts (12%) were identified after a week in culture (FIG. 3). Forty-four embryos were transferred into progestin-synchronized recipients, and 8 of the 32 recipients (25%) were detected pregnant by ultrasound 40 days after transfer. Three fetuses were electively removed at days 46 to 54 of gestation (FIG. 4), whereas 6 of the remaining recipients (75%) remained pregnant by 2 months of gestation. Four of these animals aborted between days 62 and 70 of gestation, whereas two of the pregnancies continued gestation.

Three fetuses were removed by elective cesarean section at Days 46 and 54 (twins) of gestation (FIG. 4). Crown-rump lengths (CRL) were recorded at the time of removal were 2.46 cm, 4.40 cm, and 4.60 cm for fetus number 1, 2, and 3, respectively. The fetuses were evaluated for external morphology. There was no evidence of gross external abnormalities including duplication of structures or tissues or other defects in any of the fetuses. The body of each fetus consisted of a head, trunk, limb buds or limbs and tail. Normal development appeared to be occurring in the fetuses as evidenced by the presence of a well-defined presumptive mouth, external ears, nose, and eyes. Limb buds were present in fetus number 1. For fetuses number 2 and 3, the appendicular tissues appeared normal and included proportional limbs, each with two digits at the distal end (hoof) as well as two dewclaws. These observations suggest that external development of these two fetuses was complete for early gestation.

Fibroblast cell strains were derived from the cloned animals and subjected to microsatellite marker and cytogenetic analyses. Within the family Bovidae, the domestic cattle and many other members have a normal diploid chromosome number of 60 (FIG. 2a), whereas the gaur is unique with a chromosome complement of 58 (FIG. 2b; Riggs et al, 1997; Bongso and Hilmi, 1988).

Cytogenetic analysis on the cloned cell strains revealed a normal karyotype with a modal chromosome number of 58 (FIGS. 2c-e). A large majority of the cells evaluated from each fetus were within the modal number (89-92%). Microsatellite analysis of the bovine chromosome 21 also confirmed that all three fetuses had gaurus nuclear background (FIG. 6).

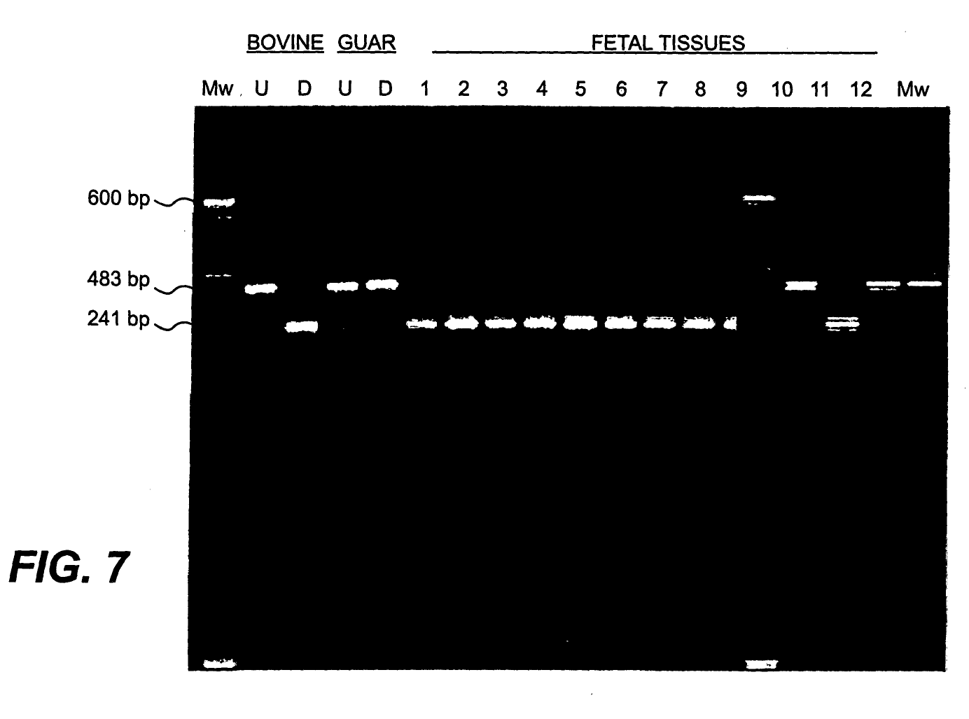

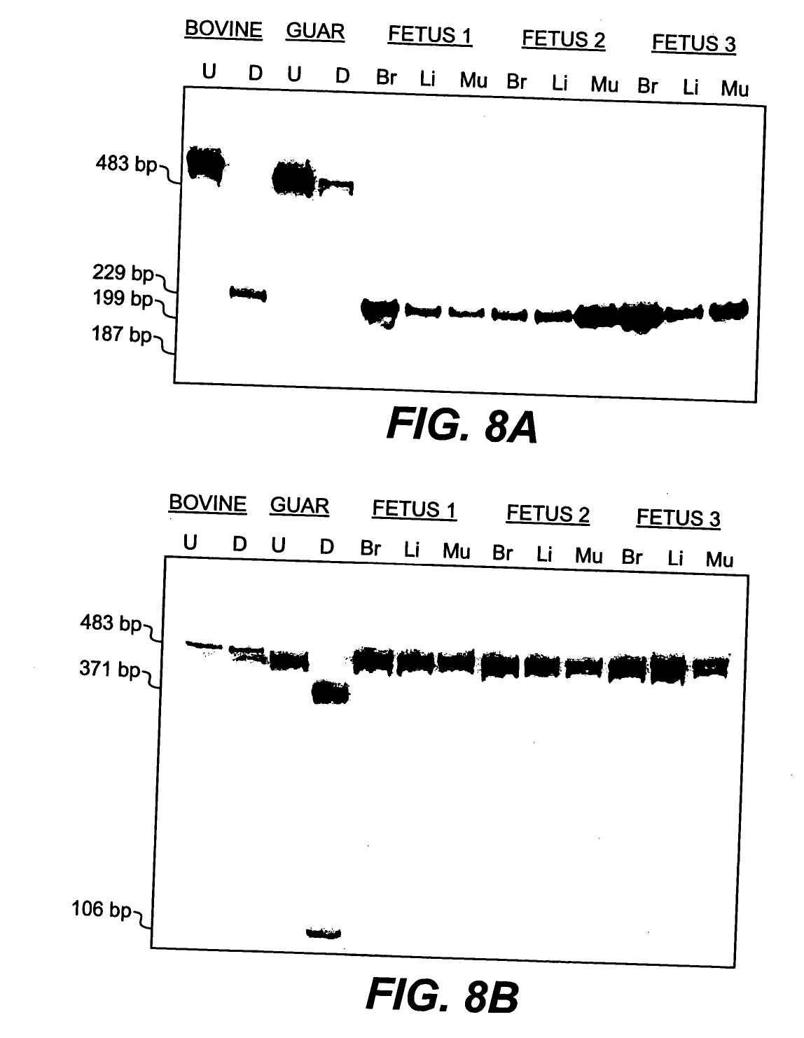

The origin of mitochondrial DNA (mtDNA) in the nuclear transfer-derived fetuses was determined by the analyses of polymorphic markers. MtDNA from the 11 different tissue types tested (brain, liver, muscle, eye, gonad, heart, intestine, lung, skin, tongue, and kidney) was exclusively taurus (FIG. 7). No gaurus mtDNA could be observed in tissues from any of the three fetuses using two different restriction fragment length polymorphisms (FIGS. 8a and b). The use of allele-specific PCR confirmed the PCR/RFLP results showing exclusively taurus mtDNA (not shown). We estimate the PCR/RFLP assay to be able to detect down to 1% of gaurus mtDNA. Serial ten-fold dilutions of purified gaurus DNA templates also showed that the allele-specific PCR has a detection limit of approximately 1% of gaurus mtDNA. There are approximately 2-5×10³ molecules of mtDNA in a somatic cell (Evans et al, 1999) as compared to approximately 1×10⁵ molecules in an oocyte (Piko and Taylor, 1987). Therefore, if every molecule of donor mtDNA had survived and replicated equally, there should be approximately 2-5% gaurus mtDNA in the nuclear transfer-derived tissues. The fact that there was no detectable contribution from the donor gaur cells is consistent with results obtained in sheep produced by intraspecific nuclear transfer. The mtDNA of each of ten cloned sheep, including Dolly the first animal cloned from an adult somatic cell, was exclusively oocyte-derived, even though nuclear transfer was also performed by whole cell fusion of somatic cells with enucleated oocytes (Evans et al, 1999).

One fetus and a partial placental unit were recovered following a late term abortion at 202 days of gestation. Crown-rump length and body weight of the male fetus was recorded and were 63.5 cm and 10.7 kg, respectively. The fetus was evaluated for overall external morphology and skeletal and internal organs were measured. As with the earlier recovered fetuses, external skeletal and internal organs appeared to be normal for its gestational age and there was no evidence of external or internal abnormalities.

Cranial development appeared normal with a head circumference of 37.1 cm. Tactile hair was present on the chin; both the ears and eyes were well defined. The eyelid had begun to separate and eyelashes were present. Development of the body and appendages were within normal limits for in vitro produced embryos (Farm and Farin, 1997). Hearthgirth was 47.0 cm and the forelimbs were proportional with the right metacarpus and metatarsus measuring 9.1 and 9.6 cm, respectively. The right hip-fetlock length was 29.0 cm and the circumferences of the fetlock and pastern were 10.2 and 8.9 cm, respectively.

All limbs contained very defined hooves and dewclaws that had started to harden. The scrotum was present and descent of the testes had occurred and tail tip hair was also observed. The weights of the internal organs, including the heart, liver, lungs and kidneys, were within normal limits (72.86, 215.9, 134.1 and 110.5 g, respectively). The animal appeared to be following a normal developmental course and the failure of the pregnancy was likely due to placentation. Gross examination of the placental tissue suggested a reduced number of cotyledons. These findings are consistent with previously published reports for cloned animals (Galton et al, 1998; Garry et al, 1996; Keefer et al, 1994; Renard et al, 1999; Solter and Gearhart, 1999; Stice et al, 1996; Wilson et al, 1995). Nuclear (FIG. 7) and mitochondrial DNA analysis by allele-specific PCR (not shown) confirmed the results observed in the previous three fetuses.

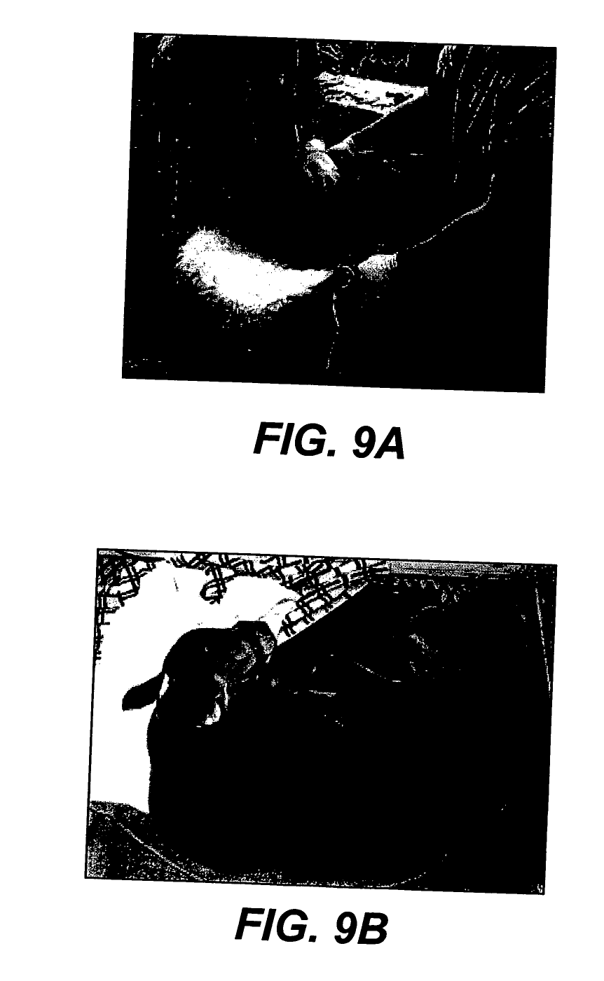

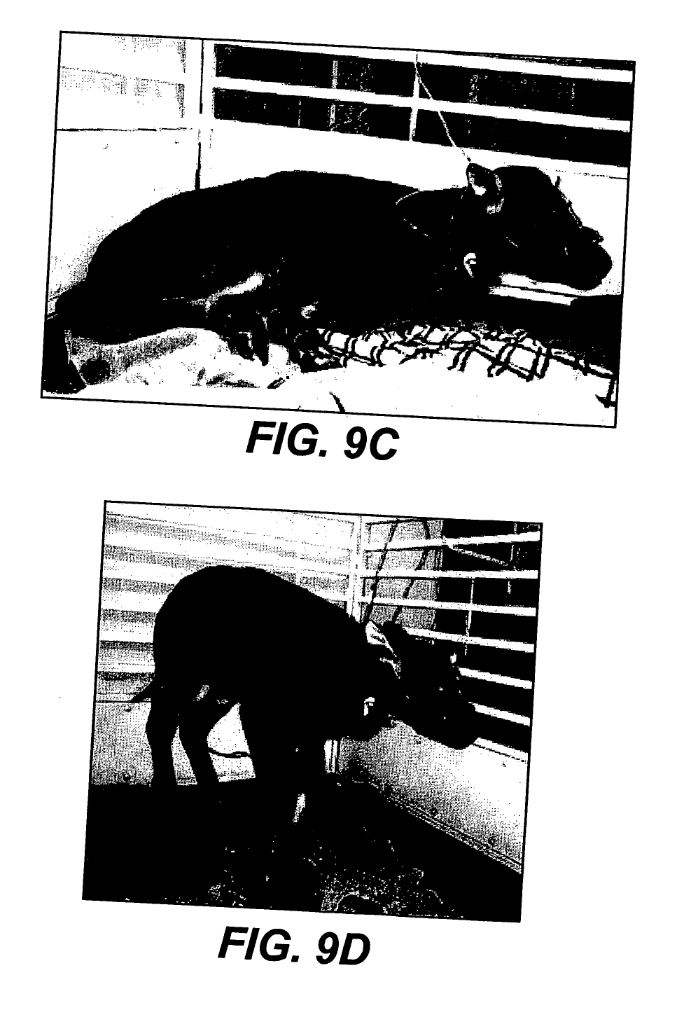

The final pregnancy was monitored very carefully and progesterone levels were monitored daily as the time of gestation neared. The levels of progesterone were slightly higher than control cows, but dropped significantly when the parturition was induced. The pregnancy was allowed to carry to 293 days of gestation at which time a caesarean section was performed. The bull calf (“Noah”) was delivered on Jan 8, 2001 at approximately 7:30 pm CST. See FIG. 9. The calf weighed 36.2 kg and was placed on minimal support therapy. Oxygen therapy was administered as a standard procedure for cloned calves. The bull calf was active, and moving shortly after birth. See FIG. 9D. He was standing and moving on his own accord by at 12 hours. The calf showed signs of sickness, diarrhea at 36 hrs post birth and was deceased by 48 hours, despite supportive therapy. The initial autopsy results suggested that there were no gross abnormalities with the calf and cause of death was a result of Clostridium perfringens Type A. There are currently no vaccines or antitoxin available for this bacterium.

The bull calf was given colostrum from one of the Holstein heifers that had recently calved at Trans Ova Genetics Genetic Achievement Center. This colostrum was used as the animal was raised under their biosecurity regulations and had a proper vaccination history. Aerobic and anaerobic bacterial cultures were negative on this colostrum. Immunoglobulin levels on this colostrum indicated a low to moderated amount of IGG1 (2860 mg/dl) and normal parameters are 3750-4750 mg/dl. However, blood levels taken on the calf at Day 1 indicated that there was passive immunity from the colostrum. Immunoglobulin levels in the blood at Day 1 were 2800 mg/dl. Failure of passive immunity occurs when levels fall below 1600 mg/dl.

Currently, we have a twin pregnancy that is at approximately 80 days of gestation.

Discussion

The cloning of an animal with the nuclear genome of one species, and the mitochondrial genome of another species has not been previously reported. It has been shown that during intraspecific crosses of mice, the paternal mtDNA can be maintained in the offspring (Kaneda et al, 1995). Microinjected Mus spretus mitochondria into Mus musculus oocytes were also maintained in a heteroplasmic state in mice (Irwin et al, 1999). Other studies however, showed that interspecific paternal mtDNA was selected against during early murine development (Shitara et al, 1998). Similarly, interspecific mtDNA was not preferentially replicated following embryonic nuclear transfer between subspecies of cattle, in which blastomeres from Bos taurus indicus embryos were fused to Bos taurus taurus oocytes (Meirelles et al., 1999). In addition, based on somatic cell experiments, we would expect a preferential maintenance of a gaurus mtDNA in the clones. In cultured cells, it was shown that the human nucleus has a strong preference for the maintenance of cognate mtDNA in cells containing both human and gorilla, or human and chimpanzee mtDNA (Moraes et al, 1999).

The fact that gaurus mtDNA was not maintained, or even amplified, in the fetuses suggests that sequence variations between gaurus and taurus mtDNA are relatively neutral at the functional level and the fusion product would behave as a fertilized egg, eliminating the exogenous mtDNA (Kaneda et al, 1995). A segment of the highly polymorphic D-loop region of mtDNA is 85% identical between taurus and gaurus, and the nucleotide sequences of the genes for cytochrome b and cytochrome oxidase subunit II are 93% and 94% identical. This high degree of identity probably results in a lack of preferential maintenance of gaurus mtDNA in the cross-species cloned fetuses. It is difficult to predict the mtDNA segregation pattern when cross-species cloning is attempted between more divergent species. These essential nuclear-mitochondrial interactions have been shown to occur, not only between different species, but also between different genera, up to approximately 8-18 million years after species radiation (Kenyon and Moraes, 1997). However, it is possible that with increasing divergence, functional problems related to nucleo-cytoplasmic compatibility could arise (Barrientos et al, 1998).

Based on the presence of key external features including cranio-facial structures (eyes, ears, mandible, tongue), as well as limbs, vertebrae, ribs, and tail in these cloned gaur fetuses; development appeared to be normal for fetuses of the Bos genus. This would imply, first, that the tissue interactions between developing hypoblast and epiblast, and subsequently between endoderm, ectoderm, and mesoderm did occur appropriately during gastrulation and neurulation in these fetuses (Noden and Lahunta, 1985). Second, these observations suggest that subsequent development of the head, face, and limbs also followed a normal pattern. Little information is available on the detailed processes involved in differentiation of external morphological structures in fetuses of the Bos genus; and, of Bos gaurus (gaur) fetuses. However, considerable information is available regarding molecular and cellular mechanisms responsible for controlling development in the other mammalian species including the mouse. The presence of apparently normal cranio-facial development present in these gaur fetuses would be consistent with the proper expression of cognates of the murine group-I aristaless-related genes (Meijlink et al, 1999).

Similarly, development of limb buds (fetus 1) and whole limbs in the fetuses is consistent with the assumption of appropriate development and interactions of the apical ectodermal ridge and progess zone for the establishment of correct patterns for proximal-distal development of the fore and hind-limb skeletal structures (Johnson and Tabin, 1997). Molecular regulation of these processes clearly would involve appropriate expression of bovine cognates of fibroblast growth factor genes including FGF-10, FGF-8, FGF-4, FGF-2, as well as cognates of sonic hedgehog (Shh), Wnt-7a, and members of the HoxA and HoxD gene families (Johnson and Tabin, 1997). The birth of the baby bull gaur, Noah, is the first successful birth of a cloned animal that is a member of an endangered species. While healthy at birth, Noah died within 48 hours from clostridial enteritis, a bacterial infection that is almost universally fatal in newborn animals. Noah's death is likely unrelated to cloning, given the showing of acceptable levels of immunoglobulin in blood samples taken on day 1. Thus, despite this setback, the birth of Noah suggests this new technology has the potential to save dozens of endangered species.

In summary, the present study provides the first evidence that mammals can be generated using interspecies nuclear transfer. Although the cloned mammals are authentic nuclear (gaur) clones, they are in fact genetic chimeras with oocyte-derived mtDNA. However, since mtDNA is transmitted by maternal inheritance, we would predict that breeding of any resultant male offspring would lead to genetically pure animals. There is also the possibility of using ‘reverse cloning’ to generate cows with gaur mtDNA as a source of oocytes for nuclear transfer. The ability to carry out successful cross-species nuclear transfer opens the way for a new strategy on the part of conservation planners to help stem the loss of valuable biological diversity and to respond to the challenge of large-scale extinctions ahead. This emerging technology also underscores the need to preserve and expand repositories of normal cell lines from species at risk of extinction.

### EXAMPLE 3

### Cloning of Extinct Species

The cells from which Noah was created originated from a male gaur that died of natural causes at 5 years of age. At autopsy, skin cells were taken and frozen and stored for eight years in the Frozen Zoo at the Center for the Reproduction of Endangered Species (CRES), at the San Diego Zoo. Eight years later the cells were thawed, and cloned using cross-species cloning. The successful cloning of endangered species from frozen cells suggests the same techniques may be used to clone species that are now extinct from frozen tissue or cell samples.

In January 2000, gamekeepers at the Spanish Ordesa National Park found the last bucardo mountain goat dead—killed by a falling tree. The bucardo mountain goat (Capra pyrenaica pyrenaica) was native to the Pyrenees mountain range in northern Spain and had a distinctive thick coat to protect it from frigid mountain air. The bucardo had been listed as an endangered species since 1973, but officials had not been able to sufficiently end the poaching and habitat destruction that eventually led to the bucardo's extinction. The Spanish government has agreed to collaborate with efforts to use interspecies nuclear transfer cloning technology to clone the bucardo from tissue retrieved and preserved before the last animal was killed.

It became apparent in the spring of 1999 that the bucardo was irreversibly headed towards extinction. At that time, Spanish biologists including Alberto Fernandez and Jose Folch took a tissue sample from the last remaining bucardo, a female, to preserve the bucardo mountain goat's cell line for the possibility of future cloning. The present inventors will take adult body (somatic) cells from the tissue and fuse them with oocytes from goats that have had their nucleus removed. The resultant embryos will be transferred into goats that will then act as surrogate mothers to the first cloned extinct animals, which will be returned eventually to their original habitat.

Future Direction

It is expected that cloning of endangered species will be at first most amenable to those species whose reproduction has already been well studied. Several zoos and conservation societies—including the Audubon Institute Center for Re-search of Endangered Species (AICRES) in New Orleans—have probed the reproductive biology of a range of endangered species, with some notable successes.

Recently, for example, Dresser and her colleagues reported the first transplantation of a previously frozen embryo of an endangered animal into another species that resulted in a live birth. In this case, an ordinary house cat gave birth to an African wildcat, a species that has declined in some areas. So far, beyond the African wildcat and the gaur, the present inventors and others have accomplished interspecies embryo transfers in four additional cases: an Indian desert cat into a domestic cat; a bongo antelope into a more common African antelope called an eland; a mouflon sheep into a domestic sheep; and a rare red deer into a common white-tailed deer. All yielded live births. The studies of felines will pave the way for cloning the cheetah, of which only roughly 12,000 remain in southern Africa. The prolonged courtship behavior of cheetahs requires substantial territory, a possible explanation for why the animals have bred so poorly in zoos and yet another reason to fear their extinction as their habitat shrinks.

One of the most exciting candidates for endangered-species cloning—the giant panda—has not yet been the subject of interspecies transfer experiments, but it has benefited from assisted reproduction technology. Following the well-publicized erotic fumblings of the National Zoo's ill-fated panda pair, the late Ling-Ling and Hsing-Hsing, the San Diego Zoo turned to artificial insemination to make proud parents of its Bai Yun and Shi Shi. Baby Hua Mei was born in August 1999. Giant pandas are such emblems of endangered species that the World Wildlife Fund (WWF) uses one in its logo.

According to a census that is now almost 20 years old, fewer than 1,000 pandas remain in their mountainous habitats of bamboo forest in southwest China. But some biologists think that the population might have rebounded a bit in some areas. The WWF expects to complete a census of China's pandas in mid-2002 to produce a better estimate.

In the meantime, strides toward the goal of panda cloning have already been made. In August 1999, Dayuan Chen and his co-workers published a paper in the English-language journal Science in China announcing that they had fused panda skeletal muscle, uterus and mam-mary gland cells with the eggs of a rabbit and then coaxed the cloned cells to develop into blastocysts in the laboratory. A rabbit, of course, is too small to serve as a surrogate mother for a giant panda. Instead, the present inventors plan to use American black bears as surrogate mothers, and are finalizing plans to obtain eggs from female black bears killed during this autumn's hunting season in the northeastern United States. Together with the Chinese, the present inventors hope to use these eggs and frozen cells from the late Hsing-Hsing or Ling-Ling to generate cloned giant panda embryos that can be implanted into a female black bear now living in a zoo. A research group that includes veterinarians at Bear Country U.S.A. in Rapid City, S.D., has already demonstrated that black bears can give birth to transplanted embryos, reporting the successful birth of a black bear cub from an embryo transferred from one pregnant black bear to another (Boone et al.).

Although cloning endangered species is controversial, it has an important place in plans to manage species that are in danger of extinction. Such plans would benefit from the establishment of a worldwide network of repositories to hold frozen tissue from all the individuals of an endangered species from which it is possible to collect samples. Those cells—like the sperm and eggs now being collected in “frozen zoos” by a variety of zoological parks—could serve as a genetic trust for reconstituting entire populations of a given species. Such an enterprise would be relatively inexpensive: a typical three-foot freezer can hold more than 2,000 samples and uses just a few dollars of electricity per year. Currently only AICRES and the San Diego Zoo's Center for Reproduction of Endangered Species maintain banks of frozen body cells that could be used for cloning.

Critics claim that the cloning of endangered species could overshadow efforts to preserve habitat. However, while habitat preservation is the keystone of species conservation, some countries are too poor or too unstable to support sustainable conservation efforts. What is more, the continued growth of the human species will probably make it impossible to save enough habitat for some other species. Cloning by interspecies nuclear transfer offers the possibility of keeping the genetic stock of those species on hand without maintaining populations in captivity, a costly enterprise, particularly in the case of large animals. Moreover, it permits the opportunity to recreate endangered and extinct species after habitat restoration is complete.

### References

Alexander, C. P. (2000) Death row. Time 155, 75-78.

Anderson, S., de Bruijn, M. H., Coulson, A. R., Eperon, L C., Sanger, F. & Young, L G. (1982). Complete sequence of bovine mitochondrial DNA. Conserved features of the mammalian mitochondrial genome. J. Mol. Biol. 156, 683-717.

Barrientos, A., Kenyon, L. & Moraes, C. T. (1998). Human xenomitochondrial cybrids. Cellular models of mitochondrial complex I deficiency. J. Biol. Chem. 5, 273:14210-14217.

Bongso, T. A. & Hilmi, M. (1988). Chromosomes of gaur cross domestic cattle hybrids Res. Vet. Sci. 44,251-254.

Boone W. R., Catlin J. C., Casey K. J., Dye P. S., Boone E. T. & Schuett R. J. (1999). Live birth of a bear cub following nonsurgical embryo collection. Theriogenology 51(3), 519-29.

Cibelli, J. B., Stice, S. L., Golueke, P. J., Kane, J. J., Jerry, J., Blackwell, C., Ponce de Leon, F. A., & Robl, J. M. (1998). Cloned transgenic calves produced from nonquiescent fetal fibroblasts. Science 280, 1256-1258.

Corley-Smith, G. E. & Brandhorst, B. P. (1999). Preservation of endangered species and populations: a role for genome banking. somatic cell cloning, and androgenesis? Mol. Reprod. Dev. 53, 363-367.

Damiani, P., Fissore, R. A., Cibelli, J. B., Long, C. R., Balise, J. J., Robl, J. M. & Duby, R. T. (1996). Evaluation of developmental competence, nuclear and ooplasmic maturation of calf oocytes. Mol. Reprod. Develop. 45, 521-534.

Dominko, T., Mitalipova, M., Haley, B., Beyhan, Z., Memili, E., McKusick, B. & First, N. L. (1999). Bovine oocyte cytoplasm supports development of embryos produced by nuclear transfer of somatic cell nuclei from various mammalian species. Biol. Repro. 60, 1496-1502.

Evans, M. J., Gurer, C., Loike, J. D., Wilmut, L, Schnieke, A. E. & Schon, E. A. (1999). Mitochondrial DNA genotypes in nuclear-transfer-derived cloned sheep. Nature Genet. 23, 90-93.

Evans, H. E. & Sach, W. O. (1973). Prenatal development of domestic and laboratory mammals: growth curves external features and selected references. Zentralbl Veterinarmed 2, 11-45.

Farin, P. W. & Farin, C. E. (1995). Transfer of bovine embryos produced in vivo or in vitro: survival and fetal development. Bio. Repro. 52, 676-682

Galton, D. J., Kay, A. & Cavanna, J. S. (1998). Human cloning: safety is the issue. Nat. Med. 4, 644.

Garry, F. B., Adams, R., McCann, J. P. & Odde, K. G. (1996). Postnatal characteristics of calves produced by nuclear transfer cloning. Theriogenology 45, 141-152.

Irwin, M. H., Johnson, L. W. & Pinkert, C. A. (1999). Isolation and microinjection of somatic cell-derived mitochondria and germline heteroplasmy in transmitochondrial mice. Transgenic Res 8, 119-123.

Johnson, R. L. & Tabin, C. J. (1997). Molecular models for vertebrate limb development. Cell 90, 979-990.

Johnston, L. A., Parrish, J. J., Monson, R., Leibfried-Rutledge, L, Susko-Parrish, J. L., Northey, D. L., Rutledge, J. J., Simmons, L. G. (1994). Oocyte maturation, fertilization and embryo development in vitro and in vivo in the gaur (Bos gaurus). J. Reprod. Fert. 100, 131-136.

Kaneda, H., Hayashi, J., Takahama, S., Taya, C., Lindahl, K. F. & Yonekawa, H. (1995). Elimination of paternal mitochondrial DNA in intraspecific crosses during early mouse embryogenesis. Proc. Natl. Acad. Sci. USA 92,4542-4546.

Keefer, C. L., Stice, S. L. & Matthews, D. L. (1994). Bovine inner cell mass cells as donor nuclei in the production of nuclear transfer embryos and calves. Biol. Reprod. 50, 935-939.

Kenyon, L & Moraes, C. T. (1997). Expanding the functional human mitochondria) DNA database by the establishment of primate xenomitochondrial cybrids. Proc. Natl. Acad. Sci. USA 94, 9131-913 5.

Lanza, R. L., Cibelli, J. B. & West, M. D. (1999). Human therapeutic cloning. Nat. Med. 5,975-977.

Lanza, R. P., Cibelli, J. B & West, M. D. (1999). Prospects for use of nuclear transfer in human transplantation. Nat. Biotech. 17, 1171-1174.

Lasley, B. L., Loskutoff, N. M. & Anderson, G. B. (1994). The limitations of conventional breeding programs and the need and promise of assisted reproduction in nondomestic species. Theriogenology 41, 119-132.

Loskutoff, N. M, Armstrong, D. L., Ohlrichs, C. L., Johnson, D. L., Funk, D.J., VanRoekel, P. V., Molina J. A., Lindsey, B. R., Looney, C. R., Bellow, S. M., Hammer, C. J., Tyler, H. D. & Simmons, L. G. (2000). Transvaginal ultrasound-guided oocyte retrieval and the development competence of in vitro-produced embryos in vitro and in vitro and in vivo in the gaur (Bos gaurus). Theriogenology 53, 337.

Meijlink, F., Beverdam, A., Brouwer, A., Oosterveen, T. C. & Berge D. T. (1999). Vertebrate aristaless-related genes. Int. J. Dev. Biol. 43, 651-663.

Meirelles, F. V., Bordignon, V., Watanabe, Y. F., Watanabe, M. R., Dayan, A., Lobo, R. B., Garcia, J. M., & Smith, L. C. (1999). Zygote reconstructions among Bos indicus and Bos taurus cattle and consequences on mitochondria) inheritance. Theriogenology 51, 209.

Moraes, C. T., Kenyon L & Hao H. (1999). Mechanisms of human mitochondrial DNA maintenance: the determining role of primary sequence and length over function. Mol. Biol. Cell. 10, 3345-3356.

Moraes, C. T., Ricci, E., Bonilla, E., DiMauro, S. & Schon, E. A. (1992). The mitochondrial tRNA(Leu(UUR)) mutation in mitochondrial encephalomyopathy, lactic acidosis, and strokelike episodes (MELAS): genetic, biochemical, and morphological correlations in skeletal muscle. Am. J. Hum. Genet. 50, 934-49.

Noden, D. M. & de Lahunta, A. (1985). The Embryology of Domestic Animals. Baltimore: Williams & Wilkins. 367pp.

Piko, L. & Taylor, K. D. (1987). Amounts of mitochondrial DNA and abundance of some mitochondrial gene transcripts in early mouse embryos. Dev. Biol. 2, 364-374 (1987).

Porter, S. (2000). Biodiversity projections look grim. World Watch 13, 8.

Renard, J. P., Chastant, S., Chesne, P., Richard, C., Marchal, J., Cordonnier, N., Chavatte, P., & Vignon, X. (1999). Lymphoid hypoplasia and somatic cloning. Lancet 353, 1489-1491.

Riggs, P. K., Owens, K. E., Rexroad, C. E., Amarai, M. E. J. & Womack, J. E. (1997). Development 22 and initial characterization of a Bos taurus X B. gaurus interspecific hybrid backcross panel. J. Heredity 88, 373-379.

Shitara, H., Hayashi, J. L, Takahama, S., Kaneda, H. & Yonekawa H. (1998). Maternal inheritance of mouse mtDNA in interspecific hybrids: segregation of the leaked paternal mtDNA followed by the prevention of subsequent paternal leakage. Genetics 148, 851-857.

Solter, D. & Gearhart, J. (1999). Putting stem cells to work. Science 283, 1468-1470.

Steinbom, R., Schinogl, P., Zakhartchenko, V., Achmann, R., Schemthaner, W., Stojkovic, M., Wolf, E., Muller, M. & Brem, G. (2000). Mitochondrial DNA heteroplasmy in cloned cattle produced by fetal and adult cell cloning. Nat. Genet. 25:255-257.

Stice, S. L., Strelchenko, N. S., Keefer, C. L., Matthews, L. (1996). Pluripotent bovine embryonic cell lines direct embryonic development following nuclear transfer. Biol. Reprod. 54, 100-110.

Stover, J., Evans, J., Dolensek, E. P. (1981). Inter-species embryo transfer from the gaur to domestic Holstein. Proc. Amer. Assoc. Zoo Vet., 122-124.

Ward, T. J., Bielawski, J. P., Davis, S. K., Templeton, J. W. & Derr, J. N (1999). Identification of Domestic Cattle Hybrids in Wild Cattle and Bison Species: A General Approach Using mtDNA Markers and the Parametric Bootstrap. Anim. Conserv. 2, 51-57.

Wells, D. N., Misica, P. M., Tervitt, H. R. & Vivanco, W. H. (1998). Adult somatic cell nuclear transfer is used to preserve the last surviving cow of the Enderby Island cattle breed. Reprod. Fertil. Dev. 10: 369-78.

White, K. L., Bunch, T. D., Mitalipov, S. & Reed, W. A. (1999). Establishrnent of pregnancy after the transfer of nuclear transfer embryos produced from the fusion of Argali (Ovis ammon) nuclei into domestic sheep (Ovis aries) enucleated oocytes. Cloning 1, 47-54.

Wilson, J. M., Williams, J. D., Bondioli, K. R., Looney, C. R., Westhusin, M. E. & D. F. McCalla (1995). Comparison of birthweight and growth characteristics of bovine calves produced by nuclear transfer (cloning), embryo transfer and natural mating. Anim. Reprod. Sci. 38, 73-83.

What is claimed:

1. A method of cloning an endangered or extinct animal, comprising:
(1) isolating a somatic cell from an endangered or extinct animal;
(2) transferring the nucleus from said somatic cell into an enucleated suitable recipient cell;
(3) activating said nuclear transfer unit;
(4) implanting said nuclear transfer unit into a suitable surrogate female; and
(5) allowing said nuclear transfer unit to develop to at least the fetal stage, thereby generating a clone of said endangered or extinct animal.

2. The method of claim 1, wherein said animal to be cloned is endangered.

3. The method of claim 2, wherein said endangered animal to be cloned is selected from the group consisting of gaur, African bongo antelope, Sumatran tiger, Giant panda, Indian desert cat, mouflon sheep and rare red deer.

4. The method of claim 3, wherein said endangered animal to be cloned is gaur, and said suitable recipient cell is an enucleated bovine oocyte.

5. The method of claim 4, wherein said suitable surrogate female is a bovine.

6. The method of claim 5, wherein said clone develops to at least the late fetal stage.

7. The method of claim 6, wherein said clone develops to at least the neonatal stage.

8. The method of claim 3, wherein said endangered animal to be cloned is an African bongo antelope, and said suitable surrogate female is an eland.

9. The method of claim 3, wherein said endangered animal to be cloned is an Indian desert cat, a Sumatran tiger, or a cheetah, and said suitable surrogate female is a domestic cat.

10. The method of claim 3, wherein said endangered animal to be cloned is a Giant panda, and said suitable surrogate female is an American black bear, and said suitable recipient cell is an enucleated American black bear oocyte.

11. The method of claim 3, wherein said endangered animal to be cloned is a mouflon sheep, and said suitable female is a domestic sheep.

12. The method of claim 3, wherein said endangered animal to be cloned is a rare red deer, and said suitable surrogate female is a common white tailed deer.

13. The method of claim 1, wherein said somatic cell is isolated from a frozen sample of cells.

14. The method of claim 13, wherein said frozen cells are semen cells.

15. The method of claim 13, wherein said animal to be cloned is extinct.

16. The method of claim 15, wherein said extinct animal to be cloned is a buccardo mountain goat of Spain.

17. The method of claim 16, wherein said suitable recipient cell is an enucleated oocyte from a domestic goat.

18. The method of claim 17, wherein said suitable surrogate female is a domestic goat.

19. A method for re-creating members of an extinct species, comprising:
(1) using nuclear transfer from frozen somatic cells to clone a male animal of an extinct species;
(2) using nuclear transfer from frozen somatic cells to clone a female animal of an extinct species; and
(3) breeding said male clone with said female clone to re-create members of said extinct species.

20. A method of producing a sexual mate of an extinct animal, comprising:
(1) isolating a somatic cell from said extinct animal;
(2) removing the sex chromosome from said somatic cell;
(3) inserting the alternative sex chromosome from a non-isogenic animal; and
(4) using nuclear transfer to create an autosomally isogenic, sexually non-isogenic sexual mate of said extinct animal.

21. The method of claim 20, wherein said alternative sex chromosome is from an allogeneic somatic cell of the same extinct species that otherwise could not be used as a nuclear transfer donor.

22. The method of claim 20, wherein said alternative sex chromosome is from a xenogeneic cell.

23. The method of claim 22, wherein said xenogeneic cell is from a species that is closely related to said extinct animal.

24. The method of claim 22, wherein said extinct animal is a female buccardo, and said xenogeneic cell is a domestic goat.

25. The method of claim 20, wherein said alternative sex chromosome is inserted via a microsome.

26. A method for re-creating members of an extinct species, comprising:
(1) using nuclear transfer from frozen somatic cells to clone an extinct animal;
(2) using the method of claim 20 to produce a sexual mate for said extinct animal; and
(3) breeding said cloned extinct animal with said nuclear transfer-generated sexual mate to recreate members of said extinct species.

27. A method of correcting chromosomal abnormalities in the cells of an extinct animal, comprising:
(1) isolating a somatic cell from a frozen sample of cells from an extinct animal;
(2) removing at least one damaged chromosome from said isolated somatic cell;
(3) inserting a functional non-isogenic chromosome into said isolated cell; and
(4) using nuclear transfer to create a partial clone of said extinct animal.

28. The method of claim 27, wherein said functional, non-isogenic chromosome is inserted via a microsome.

29. The method of claim 27, wherein said non-isogenic chromosome is isolated from a separate sample of froze cells taken from an allogeneic extinct animal.

30. The method of claim 27, wherein said non-isogenic chromosome is isolated from a xenogeneic animal, thereby creating a partial clone that is a hybrid of two species.

31. A method of cloning an extinct animal using nuclear transfer from the partial clone made by the method of claim 27.

32. An improved method for preserving and propagating an endangered species which reproduces poorly in zoos until habitat restoration is complete, comprising:
(1) isolating a somatic cell from an animal of said endangered species;
(2) transferring the nucleus from said somatic cell into an enucleated suitable recipient cell;
(3) activating said nuclear transfer unit;
(4) implanting said nuclear transfer unit into a suitable surrogate female; and
(5) allowing said nuclear transfer unit to develop into a clone of said endangered animal.

33. The method of claim 32, wherein the suitable recipient cell is from a species other than said endangered species.

34. The method of claim 32, wherein the suitable surrogate female is from a species other than said endangered species.

35. The method of claim 32 further comprising:
(6) introducing said cloned animal into said restored habitat.

36. A method of cloning an endangered or extinct animal, comprising:
(1) isolating a nucleus from a somatic cell of an endangered or extinct animal to be cloned;
(2) transferring said nucleus from said somatic cell into an enucleated suitable recipient cell;
(3) activating said nuclear transfer unit;
(4) implanting said nuclear transfer unit into a suitable surrogate female; and
(5) allowing said nuclear transfer unit to develop to at least the fetal stage, thereby generating a clone of said endangered or extinct animal.

37. The method of claim 36 wherein said nucleus is preserved in a preservative prior to nuclear transfer.

38. The method of claim 37 wherein said preservative is alcohol.

39. The cloned endangered or extinct animal produced by the method of claim 1.

40. The sexual mate of an extinct animal produced by the method of claim 20.

41. The partially cloned extinct animal produced by the method of claim 27.
